(19) United States
(12) Patent Application Publication
(10) Pub. No.: US 2012/0289534 A1
(43) Pub. Date: Nov. 15, 2012

(54) PACKAGE FOR IMPROVED TREATMENT OF CONDITIONS

(75) Inventors: Joseph PERGOLIZZI, Naples, FL (US); Maninder Chopra, Fort Myers, FL (US); Edmundo Muniz, Bonita Springs, FL (US); Neil Flanzraich, Coral Gables, FL (US)

(73) Assignee: KIRAX CORPORATION, Bonita Springs, FL (US)

(21) Appl. No.: 13/470,150

(22) Filed: May 11, 2012

Related U.S. Application Data
(60) Provisional application No. 61/485,019, filed on May 11, 2011.
(60) Provisional application No. 61/496,647, filed on Jun. 14, 2011.

(51) Int. Cl. A61K 31/485; A61P 1/12; A61P 1/08; A61J 1/00; A61P 25/20; A61P 25/34; A61P 25/06; A61P 29/00; A61P 25/04; A61P 25/22

(52) U.S. Cl. 514/282; 206/216; 206/438

(57) ABSTRACT

The present invention provides an improved package from the administration of active ingredients. The present invention provides a package comprising: a standard portion comprising one or more active ingredients in a plurality of different potencies, and a rescue portion comprising one or more same or different active ingredients. The package may be optionally be used with a patient assessment module. The present invention also provides a kit comprising: a package comprising a standard portion comprising one or more active ingredients in a plurality of different potencies, and a patient assessment module comprising instructions for administration of the standard portion. The package may optionally further comprise a rescue portion.

This application claims priority to U.S. Provisional Patent Application No. 61/485,019, filed May 11, 2011, and U.S. Provisional Patent Application No. 61/496,647, filed Jun. 14, 2011, the contents of which are hereby incorporated by reference in its entirety.

### BACKGROUND OF THE INVENTION

The majority of prescription medications are administered to a patient on a strictly regimented schedule. For example, antihypertensive agents are typically taken once or twice daily, oral contraceptives are typically taken once daily, antibiotics may be taken, e.g., once every six hours. However, there are many medications that are only taken on an as-needed basis. Examples of such medications include antitussives, analgesics, and antihistamines. It is not uncommon for health care professionals to prescribe as-needed medications for periods of time (such as, seven to thirty or more days) which exceed the actual dosing regimens required to adequately treat the condition. This results in the prescribing of a disproportionate amount of medication relative to the actual need. Prescribers may generally feel that there is a great deal of variability between patients, and therefore, to provide adequate patient care, it is necessary to prescribe medications in quantities and/or dosage strengths that exceed the typically necessary dose regimen. Alternatively, sometimes prescribers are unaware of the changes in the standard of care that update the dosing regimen recommended to treat a certain condition and therefore incorrectly prescribe doses or dosing periods for medications. A problem that can arise in these instances is that the patient is at risk for either under-dosing or over-dosing, as it can be difficult for the patient to determine his or her actual need for the medication.

In addition, many as-needed medications are potentially addictive and overprescribing can result in dependency, drug abuse and additional or excessive side effects, such as gastrointestinal bleeding or kidney damage. Some examples of medications which are typically susceptible to abuse or excessive prescribing include, but are not limited to analgesics (e.g., opioids, NSAIDs, and acetaminophen), hypnotics, and anti-anxiety agents. The availability of extra, unused dosage forms of such medications creates the potential for inappropriate, non-medical use or abuse by others through a variety of means. Further, the improper disposal of unused medicines can result in their accumulation in public utilities such as water and land, which contributes to a growing environmental hazard.

There is a need in the art for a product which provides one or more active ingredients in an appropriate dosing regimen which provides necessary and sufficient coverage to the patient for certain conditions, with an additional component, such as additional rescue dosages and/or a patient assessment module for self-evaluation, to insure adequate patient care. The present invention addresses this long felt need in the art by offering patients a predetermined dosing regimen with an additional component to enable the patient to administer an appropriate, necessary, and sufficient dose quantity and frequency for his or her particular medical need. This can be achieved through components such as rescue dosage forms and/or a self-assessment module, and allows for patients to adjust their dosing regimen. The present invention may reduce the risk of unnecessary overdose, abuse, addiction, or side effects and may allow for proper monitoring of administration to ensure safe use and patient adherence and compliance to a prescribed dosing regimen. It is a further object of the present invention to provide a dosing regimen to facilitate disease management, wherein the combination of the a standard dosing regimen and an additional component, such as rescue dosage forms and/or a patient assessment module, allows for a comprehensive or holistic (multimodal) approach to manage medical conditions. At the current time, unlike hospitalized (inpatient) patients, ambulatory (outpatient) patients do not routinely use a standard scheduled assessment of their symptoms.

The present invention may also provide convenience and assurance for patients, caregivers, and healthcare professionals who may be reluctant to administer or prescribe certain pharmaceutical products prone to abuse. By delivering a number of dosages that is appropriate for treatment (and not excessive), fewer dosages are dispensed into the community, reducing the likelihood that these dosages will be overused misused, abused, diverted or result in pollution of the water or land supply due to improper disposal. In addition, patient adherence and compliance to the prescribed dosing regimen are enhanced and the unnecessary accumulation of expired medicines is reduced. Embodiments of the present invention are expected to result in effective pharmacotherapy with decreased dosages and decreased side effects. In addition, in some embodiments of the present invention, it is expected that the amount of waste is reduced, as patients will not have a surplus of unneeded medication.

### SUMMARY OF THE INVENTION

The present invention provides a package comprising: a standard portion comprising one or more active ingredients, and a rescue portion comprising one or more same or different active ingredients. Preferably, the standard portion comprises a plurality of different potencies of one or more active ingredients. The package optionally may be used with a patient assessment module.

The present invention also provides a kit comprising: a package comprising a standard portion comprising a plurality of sections comprising a plurality of different potencies of one or more active ingredients, and a patient assessment module comprising a device configured for subjective assessment of the patient's condition and optionally correlated to subsequent administration of at least the standard portion. Optionally, the package may further comprise a rescue portion.

### BRIEF DESCRIPTION OF THE FIGURES

FIG. 1 depicts a package with dosage units arranged in a tapered manner according to an embodiment of the present invention.

FIG. 2 depicts a patient assessment module according to one embodiment of the present invention.

FIG. 3 depicts a patient assessment module according to another embodiment of the present invention.

FIG. 4 depicts a kit comprising a package with a standard portion and rescue portion, a patient assessment module comprising a self-assessment card for the patient's subjective assessment of his or her pain score, and instructions correlating the assessment to subsequent administration of the standard portion and/or the rescue portion, according to another embodiment of the present invention.

### DESCRIPTION OF THE INVENTION

The present invention provides a package comprising: a standard portion comprising one or more active ingredients, preferably in a plurality of sections comprising a plurality of different potencies of the one or more active ingredients. The present invention also provides a kit comprising: a package comprising a standard portion comprising a plurality of sections comprising a plurality of different potencies of one or more active ingredients, and a patient assessment module comprising a device configured for subjective assessment of the patient's condition and optionally correlated to subsequent administration of at least the standard portion.

In some embodiments, the package comprises a blister pack or foil pack or any other container to house the one or more active ingredients. Preferably, the package comprises a blister pack or foil pack. The package may be in any shape, including but not limited to a cylinder, oval, rectangle, circle, square, triangle, diamond, or hexagon, or any other shape which would be appropriate to house dosage forms of active ingredients. In packages which are blister packs or foil packs, the package may comprise a base layer and a barrier layer. The base layer may be made with a plastic, a plastic laminate or paper laminate, a metal foil laminate or combinations thereof. Plastics suitable for the base layer may comprise, for example, PVC, polyamide, polyolefin, polyester or polycarbonate material. The barrier layer may comprise a metal, a ceramic, or a combination thereof, such as aluminum, silicon or mixtures thereof.

The standard portion of the package is divided into a plurality of (i.e., two or more) sections. The sections, in total, comprise one or more active ingredients in at least two different potencies. Different potencies may be presented as different dosages of the same active ingredient, or in embodiments wherein more than one active ingredient is used, the different potencies may be presented as a different intensity or drug effect. For example, it is well-known that different opioids have different intensities, as well as the correlation of the relative intensities. See, e.g. Patanwala et al., Opioid Conversions in Acute Care. Ann Pharmacother. 2007; 41(2): 255-266, incorporated by reference. Thus, the present invention contemplates substituting one active ingredient for another active ingredient within a section or in a different section of the package to reduce or increase the drug intensity effect. In some embodiments, each section of the standard portion comprises one or more active ingredients in a different potency, and therefore no two sections comprise active ingredients in an identical potency. In other embodiments, there may be two or more sections among the plurality which comprise active ingredients in the same potency.

Each of the multiple sections presents an amount of the active ingredient(s) intended to be administered over a predetermined time period, for example, a number of minutes, hours, days, weeks, or months. In some preferred embodiments, the predetermined time period comprises one year, one month, one week, or one day, preferably one week or one day, and more preferably, one day. The sections of the standard portion may represent the same or different time periods of administration. For example, one section may represent an amount of active ingredient(s) intended to be administered in one day, and another section may represent an amount of active ingredient(s) intended to be administered in two days. Preferably, each of the sections represents the same intended time period of administration, most preferably one day.

In some embodiments, each of the sections in the standard portion may contain the same active ingredient, and one section may comprise a total dosage of the active ingredient that is the higher or lower than the total dosage in another section. The total dosage may be presented as different amounts of the same unit dosage form, as the same amounts of different unit dosage forms, or different amounts of different unit dosage forms. For example, one section may comprise ten 100 mg tablets of an active ingredient, and another section may comprise eight 100 mg tablets of the same active ingredient. In another example, one section may comprise three 50 mg tablets of an active ingredient, and another section may comprise three 25 mg tablets of the same active ingredient. In yet another example, one section may comprise one 100 mg tablet of an active ingredient, and another section may comprise three 25 mg tablets of the same active ingredient.

In some embodiments, sections may comprise different active ingredients, wherein one section may comprise an amount of one active ingredient which is higher or lower in intensity or drug effect, compared to that of another section. For example, one section may comprise five 2 mg tablets of hydromorphone, and another section may comprise eight 10 mg tablets of oxycodone. In another example, one section may comprise five 5 mg tablets of hydrocodone, and another section may comprise five 500 mg tablets of acetaminophen. In embodiments where a section comprises more than one active ingredient, the total intensity or drug effect of one section may differ from that of another section. For example, one section may comprise five 2 mg hydromorphone tablets and three 10 mg oxycodone tablets, and another section may comprise five 325 mg acetaminophen tablets and two 5 mg hydrocodone tablets.

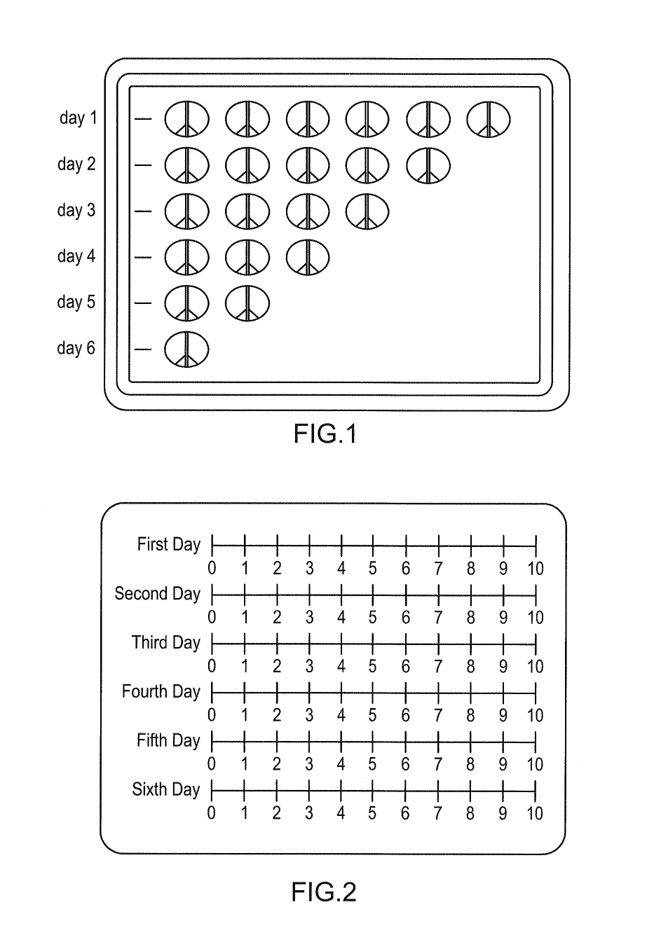

In some embodiments, the sections are arranged in decreasing potency of active ingredient(s). An example of such an arrangement of the standard portion is seen in FIG. 1, which shows decreasing amounts of the same unit dosage form over a six day period. For example, in some embodiments, one section of the standard portion may comprise active ingredient(s) of a certain potency, and the adjacent section (which is labeled to be administered over the next time period) comprises active ingredient of a lower potency. In some embodiments, such as in FIG. 1, the decreasing potency is sequential, and each adjacent section comprises active ingredient in a potency which is lower than that of a preceding section. For example, one section may comprise ten 15 mg tablets of morphine, the adjacent section may comprise eight 15 mg tablets of morphine, the next adjacent section may comprise five 15 mg tablets of morphine, and the last section may comprise three 15 mg tablets of morphine. In some other embodiments, two or more consecutive sections may comprise active ingredient(s) of the same potency, and one or more section(s) adjacent these two or more sections comprises active ingredient(s) of a different potency. For example, two adjacent sections may each comprise five 100 mg tablets of meperidine, the adjacent section may comprise three 100 mg tablets of meperidine, and the next two adjacent sections may comprise three 50 mg tablets of meperidine.

In some embodiments, the decreasing potency of active ingredient(s) may be achieved by unit dosage forms having different release profiles. In such embodiments, the package may comprise unit dosage forms of different pharmacokinetic release profiles. For example, the package may comprise immediate-release unit dosage forms and/or extended release dosage forms and/or delayed release dosage forms. In these embodiments, the amount of levels of active ingredient in the body of the patient may be tapered over a period of time, as for example, the patient may have a bolus dose of active ingredient with an immediate-release dosage form, following by lower levels of active ingredient following the administration of a extended-release dosage form after a period of time. In some embodiments, the package may comprise instructions for administration. In some embodiments, the instructions may instruct the patient on dose and frequency of administration. In some embodiments, the package is configured to provide different frequencies of administration. For example, in one embodiment, the package may comprise instructions indicating that for a first time period of administration (for example, the first day of treatment, which relates to the first section in the standard portion), a patient should administer one tablet every 4 hours, and during the next time period of administration (for example, the second day, which relates to the second section in the standard portion), a patient should administer one tablet every 6 hours.

In some embodiments, the package further comprises unit dosage forms of a placebo or inert dosage form which has no pharmacological activity. In some embodiments, the package may comprise one section (for example, within the standard portion) which contains both active ingredient and placebo. The placebo dosage forms may be separate from the active ingredient(s) dosage forms, or they may be interspersed with the active ingredient(s) dosage forms. In other embodiments, the package may comprise a section comprising only placebo.

The package of the present invention may comprise a standard dosing regimen for a patient for a designated disease, condition or indication, which can include any impairment of health or a condition of abnormal functioning. The package may be tailored or directed to certain indications or conditions. For example, a package may be created specifically for pain management following orthopedic surgery, or for pain management following dental surgery. In some embodiments, the package may be created specifically based on pain scores. For example, a package created for treatment of pain above a certain pain score, such as 5, may comprise different types and/or dosages of active ingredient(s) compared to a package created for treatment of pain associated with another pain score (for example, below 5. In addition, the package may be tailored to different patient populations. For example, a package may be created specifically for “special populations,” such as geriatric patients or for pediatric patients, as the dosing for these patient populations typically differs compared to the general population. In addition, the package may be created for specific groups, such as the visually impaired, and different language-speaking populations. In embodiments of the package for the treatment of pain, various packages may be created based on the anticipated or perceived pain tolerance of patients. For example, a package created for a patient with a low tolerance for pain may comprise a higher dosage of active ingredient(s) or more potent active ingredient(s) compared to a package created for a patient with a high tolerance for pain. The term “patient” includes any animal species, preferably mammals such as domesticated animals and humans, more preferably humans. The term “condition” refers to a variety of health states and is meant to include disorders or diseases caused by any underlying mechanism or disorder, injury, and the promotion of healthy tissues and organs. The condition or indication may be acute, chronic, or sub-chronic. The package preferably relates to the usual, recommended, or guideline administration regimen for a condition or indication. Examples of conditions or indications include but are not limited to pain, anxiety, allergies, migraines, nausea, vomiting, diarrhea, insomnia, smoking cessation, addiction, allergies, infections, sinus conditions, psychiatric conditions (such as anxiety, depression, post-traumatic stress disorder, bipolar syndrome, and schizophrenia), diabetes, cardiovascular problems, endocrine disorders, hormone, blood disorders (such as hemophilia),hormone replacement therapy, muscular dystrophy, and cystic fibrosis. In some preferred embodiments, the package relates to the treatment of pain, such as back pain, and pain associated with surgical procedures, including but not limited to urological surgery, dental procedures, hip surgery, knee-related surgery, plastic surgery, other specific orthopedic surgeries, various ambulatory and outpatient procedures, cancer surgery, and gynecological procedures such as laproscopic hysterectomy and endometriosis surgery.

In some embodiments, the package further comprises a rescue portion comprising one or more active ingredients. The active ingredient(s) of the rescue portion may be the same or different than the active ingredient(s) in the standard portion. The rescue portion may further comprise a placebo. In some embodiments, the active ingredient(s) of the rescue portion may comprise supplemental dosage forms for administration for patients who are not adequately treated with the active ingredient(s) of the standard portion. For example, in one embodiment, if a patient receives inadequate pain relief from the active ingredient(s) in the standard portion, the rescue portion may be used to provide additional pain relief. In another embodiment, if a patient is not receiving adequate glucose control from the antidiabetic medication in the standard portion, the rescue portion may be used to provide the additional medication needed to achieve adequate control. In some embodiments, the rescue portion may comprise one or more active ingredients which enhance the activity, provide a synergistic effect, or decrease the side effects of the active ingredient(s) in the standard portion. For example, in one embodiment, the standard portion may comprise an opioid pain medication, and the rescue portion may comprise a laxative or stool softener to be administered to the patient if he or she experiences constipation, which is a common side effect seen with opioids. The rescue portion may be provided in a single section intended to be administered over a single time period, or the rescue portion may be provided in multiple sections intended to be administered as needed over multiple time periods. The package may comprise instructions for administration of the rescue portion.

The active ingredients in the package are preferably contained in unit dosage form. Examples of orally administrable unit dosage forms include but not limited to a tablet, a capsule, gelcaps, a powder that can be dispersed in a beverage, a vial, ampule, or other container of liquid such as a solution or suspension, an orally disintegrating tablet, a troche, a lozenge, a lollipop, a gum, and medicated swabs. Additional examples of unit dosage forms include but are not limited to inhalers, aerosols, packages of powder or liquid to be used with inhalers or aerosols, injectables, creams, gels, lotions, ointments, balms, eye drops, ear drops, suppositories, and patches. In some preferred embodiments, the unit dosage form is a tablet or capsule. The unit dosage forms of the present invention may be immediate-release dosage forms, extended-release dosage forms, delayed-release dosage forms, depending on the type of treatment. The package of the present invention may also comprise unit dosage forms having different release profiles. For example, the package may comprise some unit dosage forms which are immediate-release dosage forms, some unit dosage forms which are extended-release dosage forms, and/or some unit dosage forms which are delayed-release dosage forms. In some embodiments, unit dosage forms may comprise different extended-release dosage forms with different release profiles. For example, the unit dosage forms may comprises extended-release dosage forms which are configured to release the active ingredient over a 12 hour period and additionally comprise extended-release dosage forms which are configured to release the active ingredient over a 6 hour period. In other embodiments, the unit dosage forms may comprise one or more extended-release dosage forms which are configured to release the active ingredient over a plurality of days, preferably to mimic the release profile (increasing and/or decreasing potency) and/or other attributes described herein of a package in accordance with the invention.

In some embodiments, the unit dosage forms may comprise different delayed-release dosage forms with different release profiles. For example, the unit dosage forms may comprises delayed-release dosage forms which are configured to begin releasing the active ingredient after a 2 hour lag time and additionally comprise delayed-release dosage forms which are configured to release the active ingredient after a 4 hour lag time. In some embodiments, the unit dosage forms may comprise immediate dosage forms and also delayed-release dosage forms and/or extended release dosage forms. In some embodiments, the unit dosage form may be one tablet or capsule, which comprises an extended-release core surrounded by an immediate release layer.

The unit dosage forms may be arranged in the package in any manner. In preferred embodiments, the package provides a visual display of the dosages to be taken. The package may comprise any quantity of unit dosage forms and may comprise one or more types of unit dosage forms.

The active ingredients in the present invention may be any ingredient, component or constituent having a pharmacological or therapeutic effect. The active ingredient may be any active agent suitable to treat, prevent, reduce the occurrence of, and reduce the symptoms related to a medical condition or indication. In some preferred embodiments, the active ingredient is a medication that is to be administered on an “as needed” basis, such as pain medications.

An active ingredient of the present invention may comprise an antihistamine. Examples of antihistamines include, but are not limited to brompheniramine maleate, chlorpheniramine maleate, carbinoxamine maleate, clemastine fumarate, dexchlorpheniramine maleate, diphenylhydramine hydrochloride, azatadine maleate, diphenhydramine citrate, diphenhydramine hydrochloride, diphenylpyraline hydrochloride, doxylamine succinate, promethazine hydrochloride, pyrilamine maleate, tripelennamine citrate, triprolidine hydrochloride, acrivastine, loratadine, desloratadine, brompheniramine, dexbropheniramine, fexofenadine, cetirizine and montelukast.

An active ingredient of the present invention may comprise an antitussive. Examples of antitussives include, but are not limited to, benzonatate, caramiphen edisylate, menthol, dextromethorphan hydrobromide and chlophedianol hydrochloride.

An active ingredient of the present invention may comprise an expectorant. Examples of expectorants include, but are not limited to, guaifenesin, ipecac, potassium iodide and tenpin hydrate.

An active ingredient of the present invention may comprise an analgesic. The analgesic may include, for example, an analgesic/antipyretic, an NSAID, an opioid, or a combination there of. Examples of analgesics include, but are not limited to, salicylates, phenylbutazone, indomethacin, phenacetin, aspirin, acetaminophen, ibuprofen, ketoprofen, diflunisal, fenoprofen calcium, flurbiprofen sodium, naproxen, tolmetin sodium, indomethacin, celecoxib, valdecoxib, parecoxib, rofecoxib, fentanyl, hydromorphone, meperidine, morphine, oxycodone, oxymorphone, tapentadol, codeine, dihydrocodeine, hydrocodone, buprenorphine, acetaminophen, tramadol, duloxetine, gabapentiods, and tricyclic antidepressants (TCAs).

An active ingredient of the present invention may comprise an antimigraine medication. Examples of antimigrane medications include, but are not limited to, sumitriptan succinate, zolmitriptan, valproic acid and eletriptan hydrobromide.

An active ingredient of the present invention may comprise an H2 receptor antagonist. Examples of H2 receptor antagonists include, but are not limited to, cimetidine, ranitidine, famotidine, and nizatidine.

An active ingredient of the present invention may comprise an proton-pump inhibitors. Examples of proton-pump inhibitors include, but are not limited to, omeprazole, lansoprazole, dexlansoprazole, pantoprazole, and rabeprazole.

An active ingredient of the present invention may comprise anti-infectious agent, such as antibiotic or anti-viral agent.

An active ingredient of the present invention may comprise a probiotic.

An active ingredient of the present invention may comprise a sedative. Examples of sedatives include, but are not limited to, trazodone, zolpidem, zaleplon, eszopiclone, nitrazepam, temazepam and melatonin.

An active ingredient of the present invention may comprise an opioid receptor antagonist. Examples of opioid receptor antagonists include, but are not limited to, methadone and naltrexone.

An active ingredient of the present invention may comprise a nicotine replacement medication or a hormone replacement medication.

An active ingredient of the present invention may comprise an antiemetic. Examples of antiemetics include, but are not limited to, scopolamine, meclizine, diphenhydramine, dronabinal, nabilone, granisetron, ondansetron, palonosetron, chlorpromazine, prochlorperazine, promethazine, metoclopramide, trimethobenzamide and apreitant.

An active ingredient of the present invention may comprise an anxiolytic. Examples of anxiolytics include, but are not limited to, alprazolam, chloriazepoxide, clonazepam, clorazepate, diazepam, estazolam, flurazepam, lorazepam, oxazepam and quazepam.

An active ingredient of the present invention may comprise an antidiarrheal. Examples of antidiarrheals include, but are not limited to, bismuth subsalicylate, nitazoxanide, calcium polycarbophil, loperamide and rifaximin.

It is contemplated by the present invention that any of the above-mentioned active ingredients may be used in combination with each other, whether as separate dosage forms or as combined dosage forms (i.e., coated or layered tablets, liquids, etc.). Combinations of the active ingredients can be utilized to provide co-therapy or to ameliorate one or more side effects of one of the agents. The dosing regimen, which includes the dosing amount and the frequency of each specific active ingredient, may be constant or variable to optimally manage the specific disease or condition. Further, the dosing of one active ingredient can start and stop at different times from other active or inert ingredients. In some embodiments, specific combinations include opioid agonist and antagonists (e.g., oxycodone and naloxone); opioid agonists and stool softeners (e.g., morphine and docusate) and opioid/acetaminophen combinations such as hydrocodone/acetaminophen or oxycodone/acetaminophen along with anti-constipation agents such as opioid antagonists. In some embodiments, the antagonist (e.g., naloxone) and/or the stool softeners are in a sufficient amount to minimize opioid-induced constipation. In such an embodiment, the antagonist or stool softener can be a separate unit dosage form to the opioid or can be combined in the same formulation (for example in the same tablet). The dose of the antagonist or stool softener can be tapered or non-tapered.

In embodiments wherein acetaminophen is combined with opioids, a lower dose strength of acetaminophen may be used, such as no more than 325 mg per unit dosage form, to mitigate and limit side effects such as hepatotoxicity.

In some embodiments, a probiotic and/or H2 receptor antagonist is combined with other active ingredients to either enhance efficacy of those said agents or mitigate their side-effects. For example, probiotics may be combined with antibiotics to reduce the incidence of antibiotic-related side effects. Such side effects include, but not limited to diarrhea caused by an imbalance in the intestinal, and particularly colonic, microbiota, such as overgrowth of potentially pathogenic organisms, including but not limited to, Clostridium difficile. In some embodiments, therapeutic doses of anti-peptic ulcer agents may be combined with antibiotics for the treatment of Helicobacter pylori infections, which is known to cause peptic ulcers. In another embodiment, combinations of NSAIDs (such as naproxen) and H2 receptor antagonist (such as famotidine) or proton-pump inhibitors or antacids may be used to mitigate gastrointestinal side effects, such as bleeding and ulcers. In some embodiments, the package may additional comprise dietary supplements, including, but not limited to, inulin.

In some embodiments wherein the package is to be administered to patients with pain-related conditions, the active ingredient comprises an opioid, such as oxycodone, hydromorphone, and hydrocodone or a salt thereof. In some embodiments the package comprises a further active ingredient such as acetaminophen or a non-steroidal anti-inflammatory drug, such as ibuprofen. In some embodiments, the opioid and further active ingredient are supplied in one dosage form, such as a combination tablet. In some embodiments wherein the active ingredient comprises oxycodone, each dosage form (such as a tablet or capsule) may comprise preferably about 0.5 to 25 mg, more preferably about 2.5 to 10 mg, and most preferably 7.5 mg of oxycodone or a salt thereof. In some embodiments wherein the active ingredient comprises hydrocodone, each dosage form may comprise preferably about 1 to 20 mg, more preferably about 2.5 to 7.5 mg, and most preferably 5 mg of hydrocodone or a salt thereof. In some embodiments wherein the package comprises an opioid and a further active ingredient, the further active ingredient comprises preferably acetaminophen or ibuprofen. In some embodiments wherein the further active ingredient comprises acetaminophen, each dosage form may comprise preferably about 100 to 750 mg, more preferably about 200 to 500 mg, and most preferably 200 mg, 325 mg or 500 mg of acetaminophen or a salt thereof. In some embodiments wherein the further active ingredient comprises ibuprofen, each dosage form may comprise preferably about 100 to 1000 mg, more preferably about 150 to 600 mg, and most preferably 200 mg of ibuprofen or a salt thereof. In some embodiments, the package comprises dosage forms which each comprise oxycodone 7.5 mg and acetaminophen 325 mg, or hydrocodone 5 mg and acetaminophen 500 mg.

In an embodiment of the present invention, a package comprises:a standard portion comprising a plurality of sections comprising unit dosage forms comprising oxycodone and acetaminophen, wherein each of the plurality of sections comprises an amount of oxycodone and acetaminophen to be administered over a period of one day,
wherein the standard portion comprises:
   a first section comprising oxycodone 45 mg and acetaminophen 1,950 mg;
   a second section comprising oxycodone 37.5 mg and acetaminophen 1,625 mg;
   a third section comprising oxycodone 30 mg and acetaminophen 1,300 mg;
   a fourth section comprising oxycodone 30 mg and acetaminophen 1,300 mg;
   a fifth section comprising oxycodone 22.5 mg and acetaminophen 975 mg;
   a sixth section comprising oxycodone 22.5 mg and acetaminophen 975 mg;
   a seventh section comprising oxycodone 22.5 mg and acetaminophen 975 mg; and
   an eighth section comprising oxycodone 22.5 mg and acetaminophen 975 mg.

The package may further comprise a rescue portion comprising unit dosage forms comprising oxycodone 52.5 mg and acetaminophen 2,275 mg and/or a patient assessment module.

In an embodiment of the present invention, a package comprises:
a standard portion comprising a plurality of sections comprising unit dosage forms comprising hydrocodone and acetaminophen, wherein each of the plurality of sections comprises an amount of oxycodone and acetaminophen to be administered over a period of one day,
wherein the standard portion comprises:
a first section comprising hydrocodone 30 mg and acetaminophen 3,000 mg;
a second section comprising hydrocodone 25 mg and acetaminophen 2,500 mg;
a third section comprising hydrocodone 20 mg and acetaminophen 2,000 mg;
a fourth section comprising hydrocodone 20 mg and acetaminophen 2,000 mg;
a fifth section comprising hydrocodone 15 mg and acetaminophen 1,500 mg;
a sixth section comprising hydrocodone 15 mg and acetaminophen 1,500 mg;
a seventh section comprising hydrocodone 15 mg and acetaminophen 1,500 mg; and
an eighth section comprising hydrocodone 15 mg and acetaminophen 1,500 mg.

The package can further comprise a rescue portion comprising unit dosage forms comprising hydrocodone 35 mg and acetaminophen 3,500 mg and/or a patient assessment module.

The present invention also provides a kit comprising a package comprising a standard portion comprising a plurality of sections comprising a plurality of different potencies of one or more active ingredients, and a patient assessment module comprising a device configured for subjective assessment of the patient's condition and optionally correlated to administration of at least the standard portion. In embodiments wherein the package comprises a standard portion and a rescue portion, the patient assessment module may comprise a device configured for subjective assessment of the patient's condition and optionally correlated to administration of the standard portion or rescue portion only and/or the administration of both the standard portion and the rescue portion.

In some embodiments, the package comprising the standard portion and the patient assessment module are configured together as one piece. For example, the patient assessment may be attached to the patient assessment module. In other embodiments, the package comprising the standard portion and the patient assessment module are separate pieces that are not physically attached to one another.

The patient assessment module may provide further assurance of proper and effective treatment, as the patient or a caregiver may assess the quality and adequacy of treatment. With the patient assessment module, the subjective assessment of the patient's condition may be completed in a number of ways. For example, the patient assessment module may comprise a device to evaluate the adequacy of the treatment of the condition. In some embodiments, the device may comprise, for example, a numerical rating scale, a visual analog scale (VAS), a chart, pull tabs, or any other manner for providing assistance to the patient in helping to determine the need for a standard or rescue dose of active ingredient. The patient assessment module may comprise more than one device.

The subjective assessment of the patient's condition by a patient or caregiver is optionally correlated to subsequent administration of the standard portion and/or the rescue portion. For example, in one embodiment, based on the assessment of the condition, the patient adjusts the dosage regimen of active ingredient(s) in at least the standard portion. In embodiments wherein the package comprises a standard portion and a rescue portion, based on the assessment of the condition, the patient adjusts the dosage regimen of active ingredient(s) in at least the standard portion, or administers the rescue portion, or both. In some embodiments, based on the assessment of the condition, the patient may take more medication, take less medication, discontinue use for a specified time period, or discontinue treatment all together. In some embodiments, based on the assessment of the condition, the patient may skip a dose if he or she is not experiencing any symptoms, or the patient may discontinue therapy if symptoms have resolved. In some embodiments, based on the assessment of the condition, the patient may adjust the specific dosing, frequency, and duration of administration of the standard portion, the rescue portion, or both. The patient assessment module may provide instructions to a patient if and/or when and how they should contact their health care provider for follow-up and/or adjustment of therapy.

In some embodiments, the patient assessment module may comprise a numerical rating scale, as shown in FIG. 2, wherein patients assess and rate their therapy (such as, level of pain) on a scale of 0 to 10. In some other embodiments, the rating scale may correlate to clinical laboratory data, for example, blood glucose levels or blood pressure values. In one embodiment, wherein the patient assessment module comprises a numerical rating scale, the patient assessment module may further provide instructions for administration. For example, in one embodiment, the patient assessment module may provide instructions to discontinue therapy if the rating is 0 to 1, to administer one tablet from the standard portion if the numerical rating is 2 to 6, and to administer two tablets from the standard portion if the numerical rating is from 6 to 10. In another embodiment, for example, the patient assessment module may provide instructions to administer one or more dosages from the rescue portion if the rating is greater than 5, or to take one tablet from the rescue portion if the numerical rating does not decrease one hour after administration of a tablet from the standard portion. In another embodiment, the patent assessment module may provide instructions for how to administer the standard portion and how to administer the rescue portion. For example, the patient assessment module may provide instructions to discontinue therapy if the rating is 0 to 1, to administer one tablet from the standard portion if the numerical rating is 2 to 6, and to administer two tablets from the standard portion if the numerical rating is from 6 to 10, and to further administer one tablet from the rescue portion if the numerical rating is 8 to 10.

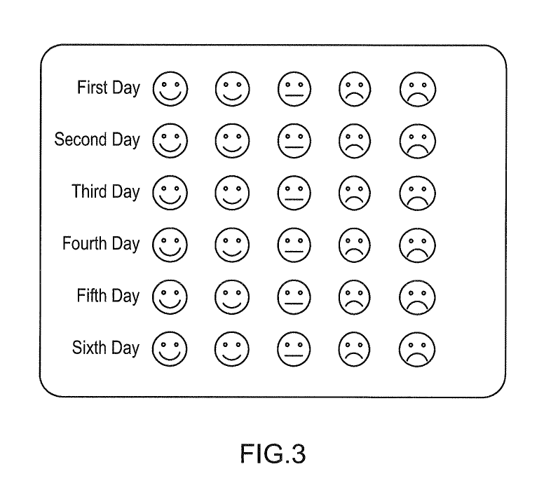

In some other embodiments, the patient assessment module may comprise a scale as shown in FIG. 3, wherein patients assess and rate their therapy by the level of satisfaction (very happy face, happy face, neutral face, sad face, and very sad face). In some other embodiments, the patient assessment module comprises a chart which provides, for example, a checklist of symptoms.

In some embodiments, the patient assessment module may be used to evaluate therapy at any time during treatment, for example, after each dose or after a number of doses, or after a time period, such as every four hours or once every day.

In some embodiments, the patient assessment module may be affixed to the package or it may be a non-attached component. For example, in some embodiments, the patient assessment module may be present on a box or container containing the package, or it may be affixed directly to the package itself. In some embodiments, the patient assessment module may also be a separate card provided with the kit, or it may be provided via a computer, for example, as an application for a mobile device, a tablet or personal digital assistant, a program for a computer, or a link to a webpage which contains, for example, an evaluation scale.

In some embodiments, the kit may comprise a “talking label,” or an electronic device which produces a digitally synthesized sound resembling human voice, or a pre-recorded human voice, educating and enabling the patient to understand the key information of the kit and package, including but not limited to, information regarding the active ingredient's efficacy, safety, and dosing regimen, and optionally, instructions regarding how to evaluate and assess their treatment and how to utilize the patient assessment module. In some embodiments, the talking label may be embedded in the package or exist as an independent device.

In some embodiments, the kit comprises a device which electronically transmits information inputted by patients in the patient assessment module to a third party, such as a healthcare provider, via the internet or a network. This information may be used by healthcare providers to determine whether any additional, follow-up interaction between healthcare provider and patient is necessary, for example, to alter the pharmacotherapy.

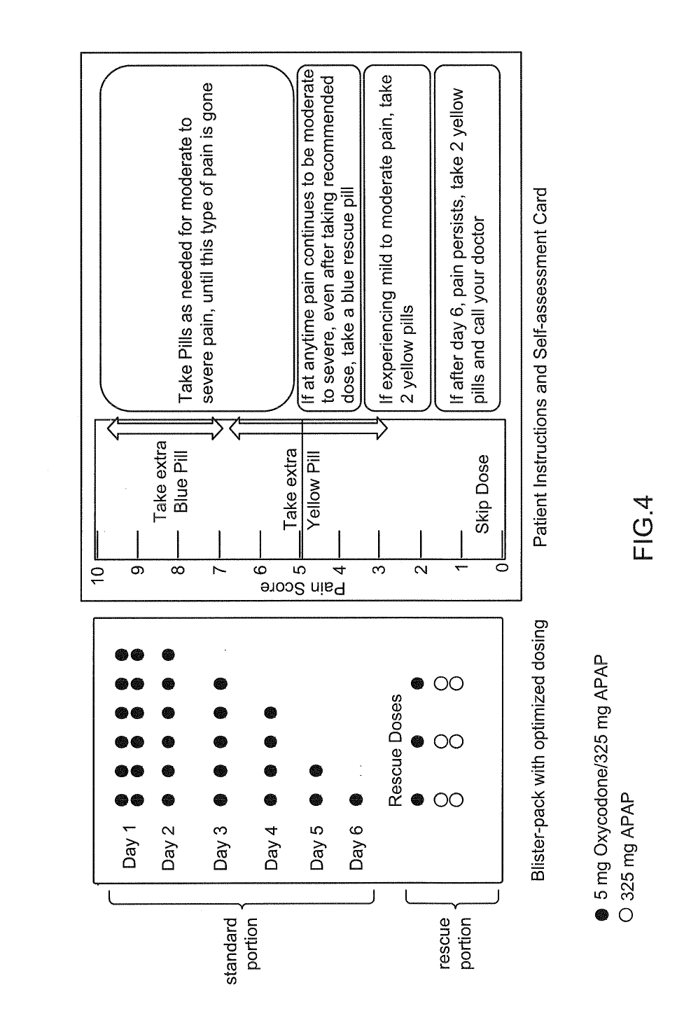

An example of a kit comprising a package comprising a standard portion and a rescue portion and a patient assessment module is seen in FIG. 4. The patient assessment module in FIG. 4 comprises a numerical rating scale for pain and instructions for administration of active ingredient in the standard portion and the rescue portion.

Methods of conducting singular or repeated measurements of systemic levels of the active ingredient (for example, a specific opioid such as oxycodone) can be employed to detect relative instances wherein the drug level is relatively lower for the same person. Patients may then be provided a recommendation to take an additional dose to prevent any impending relative severity of pain. These measurements may include direct methods to measure the levels of circulating medicine in the collected blood, or other biomarkers known to be correlated with circulating levels, such as saliva and skin.

The identification of the active ingredient(s) and the dosages of the active ingredient(s) to be included in the package can be determined by conducting a patient study to assess the appropriate dosing regimen. For example, prescription data showing the amount and type of active ingredient(s) which are prescribed by health care professionals in association with a condition or indication may be compiled. In addition, consumption data showing the actual amount of active ingredients consumed or taken by patients may be compiled. This data may be compiled by having patients record their use in a diary or journal, wherein the amount of medication and the time of consumption of medication is recorded. In some embodiments, in addition to the amount and time of medication consumption, the patent may further record objective and subjective data associated with their medication use. For example, the patient may record objective clinical data such as blood glucose levels, electrolyte levels, or any other clinical parameter. In some other embodiments, the patient may record subjective data such as pain scores, side effects, or satisfaction level with medication use. Based on the prescription data and/or consumption data, the identification of the appropriate regimen, including the active ingredient(s) and dosages of active ingredient(s) in a package, may be determined. In some embodiments wherein data on the average consumption of medication (average dosage) are compiled, a package may be created, wherein the dosages of the active ingredient(s) in the standard portion and/or the rescue portion are based on the average dosage. In some embodiments, the package may contain a higher dosage than the average dosage; for example, the package may contain a dosage that is the average dosage plus an addition of one, two or three standard deviation equivalents.

The present invention preferably provides a dosing regimen that is necessary and sufficient to alleviate the disease, symptom or condition being treated. The present invention is especially useful in disease conditions in which severity of the disease may be dynamically (or temporally) changing, wherein the dosing regimen is designed to manage and alleviate the disease condition in an optimal manner. The invention is intended to prevent or minimize under-dosing or overdosing until the disease symptoms are sufficiently and adequately alleviated.

The present invention is intended to provide convenience and assurance for patients and caregivers who may be reluctant to take or prescribe respectively certain pharmaceutical products without a clear day-over-day reduction. By delivering a limited number of dosages, fewer dosages are dispensed into the community, reducing the likelihood that these dosages will be overused, misused, abused, diverted or pollute the water supply due to improper disposal, and at the same time enhancing patient adherence and compliance to the prescribed dosing regimen, and prevent unnecessary accumulation of expired medicines. In preferred embodiments, the invention offers a visual display of the dosages to be taken; should anyone try to tamper with the pack or remove some of the pills for inappropriate use, the theft would be immediately apparent.

As disclosed above, the dosing regimen preferably is chosen such that patient's condition is adequately and satisfactorily treated. The patient assessment modules of the present invention provide further assurance of proper and effective treatment as the patient has evaluation/assessment tools which are used to determine the patient's need for a specific dose of the active agent.

Unless defined otherwise, all technical and scientific terms used herein have the same meaning as commonly understood by one of ordinary skill in the art. Although any methods and materials similar or equivalent to those described herein can also be used in the practice or testing of the present invention, the preferred methods and materials are now described. It must be noted that as used herein and in the appended claims, the singular forms “a”, “and”, and “the” include plural references unless the context clearly dictates otherwise. All technical and scientific terms used herein have the same meaning.

### EXAMPLES

The following examples are presented for the purpose of illustrating embodiments of this invention, and are not to be construed as limiting the scope of the invention in any way since, as recognized by one skilled in the art, particular agents, strengths, dosing frequencies and duration of therapy could be modified as needed for individual circumstances.

### Example 1

An embodiment of the standard portion of the package of the present invention is depicted in FIG. 1. The package comprises a plurality of sections, with each section comprising a row of unit dosage forms. Each section may contain unit dosage forms comprising the same or different active ingredients. Each section may comprise unit dosage forms of identical or differing dosage strength. The first section may comprise, for example, 100 mg unit dosage forms, while the second row may comprise 75 mg unit dosage forms and the third row may comprise 50 mg unit dosage forms, and so on. Also, in an embodiment where active ingredients are arranged in decreasing potency, there may be an equal number of unit dosage forms in each row, but each row may contain a lower dosage amount or strength than the one above it (not depicted). Alternatively, each row section may contain the same dosage amount or strength of an active ingredient, but the frequency of the dose taken daily may decrease from section to section. For example, all of the unit dosage forms in the package may be 100 mg tablets, but the patient may be instructed to take one tablet every four hours on day 1, one tablet every five hours on day 2, one tablet every six hours on day 3, and so on. It is contemplated that the frequency may also be decreased by days instead of hours. For example, the first section may contain unit dosage forms to be taken once daily for six days in Week 1 (e.g., Sunday through Friday); the second section may contain unit dosage forms to be taken once daily for five days in Week 2 (e.g., Monday through Friday); the third section may contain unit dosage forms to be taken every other day in Week 3 (e.g., Sunday, Tuesday, Thursday, and Saturday); and so on. In such an embodiment, the total amount of active ingredient administered each week preferably decreases.

### Example 2

### Hydrocodone/Acetaminophen Dose Packs

A blister pack may be prepared with a combination tablet comprising hydrocodone 5 mg and acetaminophen 500 mg (e.g., VICODIN®). The blisters are arranged in a tapered manner with Day 1 and Day 2 providing 8 tablets in total (4 per day), to provide a dose of 1 to 2 tablets every 6 hours. The blisters are arranged on Day 3 and Day 4 with 4 tablets in total (per day) to provide a dose of 1 tablet every 6 hours. The blisters are arranged on Day 5 with 3 tablets to provide a dose of 1 tablet every 8 hours. A patient assessment module may be used with the blister pack.

The same type of blister pack may be prepared with a different combination product such as hydrocodone 7.5 mg and acetaminophen 500 mg (LORTAB®) or any strength of an oxycodone/acetaminophen product (e.g., PERCOCETφ).

### Example 3

### Oxycodone/Acetaminophen Dose Packs

A blister pack may be prepared with a combination tablet comprising oxycodone and acetaminophen. The blisters are arranged in a tapered manner (by strength) with each of Day 1 through Day 5 providing dosing on an every four hour dosing interval (6 doses per day). Day 1 and Day 2 provide a product with 10 mg oxycodone and 325 mg acetaminophen. The blisters are arranged on Day 3 to provide a product with 7.5 mg oxycodone and 325 mg acetaminophen. The blisters are arranged on Day 4 to provide a product with 5 mg oxycodone and 325 mg acetaminophen. The blisters are arranged on Day 5 to provide a product with 2.5 mg oxycodone and 325 mg acetaminophen. A patient assessment module is associated with the dose pack.

In some embodiments, the oxycodone and acetaminophen may be provided as separate tablets, instead of combined in one combination tablet. Other dose packs can be prepared which taper the dosage form by both frequency of dosing and dosage strength.

### Example 4

### Further Examples of Dose Packs

EXAMPLE DOSE PACK A

| | |
|---|---|
| | # of |
| Day | units* |

STANDARD
PORTION

| | |
|---|---|
| 1 | 2 |
| 2 | 2 |

OPTIONAL RESCUE
PORTION

6

EXAMPLE DOSE PACK B

| | |
|---|---|
| | # of |
| Day | units* |

STANDARD
PORTION

| | |
|---|---|
| 1 | 3 |
| 2 | 2 |
| 3 | 2 |

OPTIONAL RESCUE
PORTION

6

EXAMPLE DOSE PACK C

| | |
|---|---|
| | # of |
| Day | units* |

STANDARD
PORTION

| | |
|---|---|
| 1 | 4 |
| 2 | 3 |
| 3 | 1 |
| 4 | 2 |

OPTIONAL RESCUE
PORTION

7

EXAMPLE DOSE PACK D

| | |
|---|---|
| | # of |
| Day | units* |

STANDARD
PORTION

| | |
|---|---|
| 1 | 4 |
| 2 | 3 |
| 3 | 2 |
| 4 | 2 |
| 5 | 2 |

OPTIONAL RESCUE
PORTION

8

| | | | | |
|---|---|---|---|---|
| EXAM- | EXAM- | EXAM- | EXAM- | EXAM- |
| PLE | PLE | PLE | PLE | PLE |
| DOSE | DOSE | DOSE | DOSE | DOSE |
| PACK E | PACK F | PACK G | PACK H | PACK I |

| | |
|---|---|
| Day | # of units* |

STANDARD PORTION

| | | | | | |
|---|---|---|---|---|---|
| 1 | 6 | 5 | 7 | 4 | 6 |
| 2 | 4 | 3 | 5 | 3 | 4 |
| 3 | 4 | 3 | 5 | 4 | 6 |
| 4 | 3 | 3 | 5 | 3 | 5 |
| 5 | 3 | 3 | 4 | 3 | 5 |
| 6 | 3 | 2 | 3 | 2 | 4 |
| 7 | 2 | 2 | 3 | 2 | 3 |

OPTIONAL RESCUE PORTION

| | | | | |
|---|---|---|---|---|
| 5 | 3 | 6 | 3 | 6 |

| | | |
|---|---|---|
| EXAMPLE | EXAMPLE | EXAMPLE |
| DOSE | DOSE | DOSE |
| PACK J | PACK K | PACK L |

| | |
|---|---|
| Day | # of units* |

STANDARD PORTION

| | | | |
|---|---|---|---|
| 1 | 7 | 8 | 6 |
| 2 | 5 | 6 | 5 |
| 3 | 4 | 6 | 4 |
| 4 | 4 | 6 | 4 |
| 5 | 3 | 5 | 3 |
| 6 | 3 | 4 | 3 |
| 7 | 3 | 4 | 3 |
| 8 | 3 | 2 | 3 |

OPTIONAL RESCUE PORTION

| | | |
|---|---|---|
| 6 | 5 | 7 |

| | |
|---|---|
| EXAMPLE | EXAMPLE |
| DOSE | DOSE |
| PACK M | PACK N |

# of units

STANDARD PORTION

| | | |
|---|---|---|
| 1 | 9 | 9 |
| 2 | 8 | 7 |
| 3 | 6 | 6 |
| 4 | 6 | 6 |
| 5 | 5 | 5 |
| 6 | 5 | 4 |
| 7 | 5 | 5 |
| 8 | 4 | 5 |
| 9 | 2 | 2 |

OPTIONAL RESCUE PORTION

| | |
|---|---|
| 7 | 7 |

** # of units: A unit may refer to: (i) a unit dosage form comprising oxycodone (2.5 mg, 5 mg, or 7.5 mg) and a unit dosage form comprising acetaminophen (200 mg, 325 mg, or 500 mg); (ii) a single unit dosage form comprising oxycodone (2.5 mg, 5 mg, or 7.5 mg) and acetaminophen (200 mg, 325 mg, or 500 mg); (iii) a unit dosage form comprising hydrocodone (2.5 mg, 5 mg, or 7.5 mg) and a unit dosage form comprising acetaminophen (200 mg, 325 mg, or 500 mg); (iv) a single unit dosage form comprising hydrocodone (2.5 mg, 5 mg, or 7.5 mg) and acetaminophen (200 mg, 325 mg, or 500 mg); (v) a unit dosage form comprising oxycodone (2.5 mg, 5 mg, or 7.5 mg); (vii) a unit dosage form comprising hydrocodone (2.5 mg, 5 mg, or 7.5 mg); (viii) a unit dosage form comprising acetaminophen (200 mg, 325 mg, or 500 mg); or (ix) a unit dosage form comprising ibuprofen (200 mg).

As demonstrated in this Example, in some embodiments, the package may comprise: 2 to 9 units on day one, and optionally, further one or more of the following: 2 to 8 units on day two, 2 to 6 units on day three, 2 to 6 units on day four, 2 to 5 units on day five, 2 to 5 units on day six, 2 to 5 units on day seven, 2 to 5 units on day eight, and 2 units on day nine.

### Example 5

### Determination of Dosing Regimen

The package of the present invention may provide a dosing regimen tailored to a certain condition or indication. The dosing regimen may be determined from clinical guidelines or any other method. For example, studies may be conducted to determine usual prescribing patterns and typical actual administration of medications.

A non-interventional observational pilot study of twenty patients was conducted to evaluate the duration of time that a discharged postoperative gynecology, urology, or orthopedic outpatient requires oral, opioid-based analgesics for the management of moderate to severe pain. Patients enrolled in the study were given subject diaries to record the following information: whether the medication was taken, the number of tablets administered, the time the tablet(s) were administered, the pain level (on a scale of 0 to 10) before the tablets were administered, any side effects experienced as a result of the medication, and any additional medication that were administered. The compilation of this data will assist in the determination of the appropriate dosing regimen for certain indications. Packages may be prepared based on the data compiled for specific conditions or indication (for example, a pain relief package for postoperative pain relating to gynecological procedures, or a pain relief package for postoperative pain for geriatric patients). An example of part of a subject diary of a patient taking a combination tablet of oxycodone 5 mg and acetaminophen 325 mg is exemplified below:

| | | Dose #1 | Dose #2 | Dose #3 |
|---|---|---|---|---|
| Day 0 | Time | 22:13 | | |
| (day of | Number of tablets taken | 1 | | |
| surgery) | Pain score | 8 | | |
| Day 1 | Time | 5:25 | 11:55 | 20:55 |
| | Number of tablets taken | 1 | 1 | 1 |
| | Pain score | 9 | 7 | 8 |
| Day 2 | Time | 2:25 | 12:45 | 20:00 |
| | Number of tablets taken | 1 | 1 | 1 |
| | Pain score | 8 | 8 | 6 |
| Day 3 | Time | 2:00 | 11:00 | 20:00 |
| | Number of tablets taken | 1 | 1 | 1 |
| | Pain score | 6 | 6 | 7 |
| Day 4 | Time | 14:00 | 22:00 | |
| | Number of tablets taken | 1 | 1 | |
| | Pain score | 7 | 7 | |
| Day 5 | Time | 22:00 | | |
| | Number of tablets taken | 1 | | |
| | Pain score | 7 | | |
| Day 6 | Time | 22:30 | | |
| | Number of tablets taken | 1 | | |
| | Pain score | 7 | | |
| Day 7 | Time | 12:00 | 22:00 | |
| | Number of tablets taken | 1 | 1 | |
| | Pain score | 7 | 6 | |
| Day 8 | Time | 22:35 | | |
| | Number of tablets taken | 1 | | |
| | Pain score | 5 | | |
| Day 9 | Time | 22:35 | | |
| | Number of tablets taken | 1 | | |
| | Pain score | 5 | | |
| Day 10 | Time | 22:45 | | |
| | Number of tablets taken | 1 | | |
| | Pain score | 5 | | |

Full patient data follows:

| | | # of pills | Total Pills | % |
|---|---|---|---|---|
| Patient (Age and race) | Prescription given | prescribed | Consumed | Consumed |
| 31 yr black male | Oxycodone 7.5 mg/APAP 325 mg | 50 | 30 | 60.0 |
| 39 year black male | Oxycodone 7.5 mg/APAP 325 mg | 50 | 19 | 38.0 |
| 35 yr caucacian male | Oxycodone 7.5 mg/APAP 325 mg | 50 | 44 | 88.0 |
| 36 yr caucacian male | Oxycodone 7.5 mg/APAP 325 mg | 25 | 7 | 28.0 |
| 37 yr caucacian male | Oxycodone 7.5 mg/APAP 325 mg | 50 | 4 | 8.0 |
| 38 yr caucacian male | Oxycodone 7.5 mg/APAP 325 mg | 25 | 20 | 80.0 |
| 31 yr caucacian male | Hydromorphone 4 mg | 50 | 46 | 92.0 |
| 30 yr caucacian male | Oxycodone 7.5 mg/APAP 325 mg | 50 | 39 | 78.0 |
| 38 yr cucacian male | Oxycodone 7.5 mg/APAP 325 mg | 50 | 11 | 22.0 |
| 35 yr black male | Oxycodone 7.5 mg/APAP 325 mg | 50 | 1 | 2.0 |
| 59 yr caucacian male | Oxycodone 5 mg/APAP 325 mg | 30 | 8 | 26.7 |
| 53 yr caucacian female | Oxycodone 5 mg/APAP 325 mg | 30 | 26 | 86.7 |
| 78 yr caucacian male | Hydrocodone 5 mg/APAP 500 mg | 30 | 7 | 23.3 |
| 51 yr caucacian female | Oxycodone 5 mg/APAP 325 mg | 30 | 14 | 46.7 |
| 61 yr caucacian female | Oxycodone 7.5 mg/APAP 325 mg | 38 | 32 | 84.2 |
| 51 yr black female | Hydrocodone 5 mg/APAP 500 mg | 20 | 16 | 80.0 |
| 66 yr caucacian female | Hydrocodone 5 mg/APAP 500 mg | 24 | 0 | 0.0 |
| 51 yr caucacian female | Oxycodone 5 mg/APAP 325 mg | 30 | 3 | 10.0 |
| 67 yr caucacian female | Oxycodone 5 mg/APAP 325 mg | 24 | 2 | 8.3 |
| 21 yr caucacian male | Oxycodone 5 mg/APAP 325 mg | 30 | 14 | 46.7 |

### Example 6

### Prophetic Examples of Studies to Determine the Effectiveness of a Dose Pack of the Present Invention

### Example 6a

An observational pilot study to determine the feasibility of an at home postoperative pain Numerical Rating Scale (NRS) assessment tool in patients undergoing outpatient surgery requiring moderate to severe analgesic medication on each day (24 hour period) after completion of surgery up to postoperative day 10.

This observational pilot study would evaluate the feasibility of a self-administered Numerical Rating Scale (NRS) home assessment of post operative pain in outpatient postoperative outpatients who require moderate to severe analgesics for the management of their pain. In the preoperative holding area, patients would be provided with a postoperative pain assessment NRS tool. Instructions would be provided on how to complete the NRS tool. The self assessment would ask the question on each postoperative day, beginning in the morning and then successively for every 6 hours until bedtime. The returned NRS assessment record and the questionnaire would be analyzed to study effectiveness of the NRS tool.

### Example 6b

Study to evaluate the efficacy and safety of a standard moderate to severe postoperative analgesia dosing regimen versus a novel tapered dose pack with a built-in assessment tool In the outpatient surgical setting

This study would evaluate the safety and efficacy of an opioid dose-pack with an assessment tool for the management of moderate to severe postoperative pain in the outpatient surgical setting (Arm A) versus standard of care (Arm B). The pilot study may enroll 20-40 patients. In the preoperative holding area patients would be instructed on the use of the assessment diary. Additionally, patients Arm A would be instructed on how and when to complete the pain numerical rating scale (NRS). Efficacy, safety profile, patient and prescriber satisfaction, ease of use related to numerical rating scale for postoperative pain, ease of use and understanding of dose pack directions, comparison of total amount of opioids used during treatment period, need for adjuvant therapy, and perceived societal benefits would be assessed. This pilot study would be the basis of an expanded randomized double blind study required by regulatory agencies.

### Example 6c

A preclinical study to titrate opioid dosing required for optimal pain relief in animal models of postsurgical pain.

A rat paw incisional model for post operative pain (BRENNAN T. J., VANDERMEULEN E. P., GEBHART G. F. Characterization of a rat model of incisional pain. Pain. 1996; 64:493-501) would be employed to find optimal dosing regimen to alleviate pain by dose titrating for 14 days.

What is claimed:

1. A package comprising:
a standard portion comprising one or more active ingredients, and
a rescue portion comprising one or more same or different active ingredients.

2. The package of claim 1, wherein the standard portion comprises a plurality of sections comprising a plurality of different potencies of one or more active ingredients.

3. The package of claim 1, wherein each of the plurality of sections comprises an amount of one or more active ingredients to be administered over a predetermined time period.

4. The package of claim 3, wherein plurality of sections is arranged in decreasing potency.

5. The package of claim 4, wherein the plurality of sections is arranged in sequentially decreasing potency.

6. The package of claim 3, wherein the predetermined time period is selected from the group consisting of one day, one week, one month, and one year.

7. The package of claim 1, wherein the package further comprises placebo unit dosage forms.

8. The package of claim 1, wherein the standard portion and the rescue portion both comprises one or more of the same active ingredients.

9. The package of claim 1, wherein the one or more active ingredients comprises an agent selected from the group consisting of: analgesics, antimigraine medications, H₂ receptor antagonists, proton pump inhibitors, sedatives, opioid receptor antagonists, nicotine replacement medications, antiemetics, anxiolytics, and antidiarrheal medications.

10. The package of claim 1, wherein the one or more active ingredients comprises opioids.

11. The package of claim 1, wherein the package comprises two or more active ingredients, wherein one of the active ingredients enhances the activity, provides a synergistic effect, or decreases the side effects of another active ingredient.

12. A kit comprising:
a package comprising a standard portion comprising a plurality of sections comprising a plurality of different potencies of one or more active ingredients, and
a patient assessment module comprising a device configured for subjective assessment of the patient's condition and optionally correlated to subsequent administration of one or more active ingredients.

13. The kit of claim 12, wherein the patient assessment module comprises a component selected from the group consisting of: numerical rating scales, visual analog scales, and charts.

14. The kit of claim 12, wherein the package further comprises a rescue portion comprising one or more same or different active ingredients.

15. The kit of claim 14, wherein the patient assessment module comprises a device configured for subjective assessment of the patient's condition and optionally correlated to subsequent administration of one or more active ingredients in the standard portion, the rescue portion, or both the standard portion and the rescue portion.

16. The kit of claim 12, wherein each of the plurality of sections sections comprises an amount of one or more active ingredients to be administered over a predetermined time period.

17. The kit of claim 12, wherein the plurality of sections are arranged in decreasing potency.

18. The kit of claim 12, wherein the plurality of sections are arranged in sequentially decreasing potency.

19. The kit of claim 16, wherein the predetermined time period is selected from the group consisting of one day, one week, one month, and one year.

20. The kit of claim 14, wherein the standard portion and the rescue portion both comprises one or more of the same active ingredients.

21. The kit of claim 12, wherein the one or more active ingredients comprises an agent selected from the group consisting of: analgesics, antimigraine medications, H₂ receptor antagonists, proton pump inhibitors, sedatives, opioid receptor antagonists, nicotine replacement medications, antiemetics, anxiolytics, and antidiarrheal medications.

22. The kit of claim 12, wherein the one or more active ingredients comprises an opioid.

23. The kit of claim 12, wherein the package comprises two or more active ingredients, wherein one of the active ingredients enhances the activity, provides a synergistic effect, or decreases the side effects of another active ingredient.

24. The kit of claim 12, wherein the package and the patient assessment module are configured together as one piece.

25. A package comprising:
a standard portion comprising a plurality of sections comprising unit dosage forms comprising oxycodone and acetaminophen, wherein each of the plurality of sections comprises an amount of oxycodone and acetaminophen to be administered over a period of one day,
wherein the standard portion comprises:
a first section comprising oxycodone 45 mg and acetaminophen 1,950 mg;
a second section comprising oxycodone 37.5 mg and acetaminophen 1,625 mg;
a third section comprising oxycodone 30 mg and acetaminophen 1,300 mg;
a fourth section comprising oxycodone 30 mg and acetaminophen 1,300 mg;
a fifth section comprising oxycodone 22.5 mg and acetaminophen 975 mg;
a sixth section comprising oxycodone 22.5 mg and acetaminophen 975 mg;
a seventh section comprising oxycodone 22.5 mg and acetaminophen 975 mg; and
an eighth section comprising oxycodone 22.5 mg and acetaminophen 975 mg.

26. The package of claim 25, wherein the package further comprises a rescue portion comprising unit dosage forms comprising oxycodone 52.5 mg and acetaminophen 2,275 mg.

27. A package comprising:
a standard portion comprising a plurality of sections comprising unit dosage forms comprising hydrocodone and acetaminophen, wherein each of the plurality of sections comprises an amount of oxycodone and acetaminophen to be administered over a period of one day,
wherein the standard portion comprises:
a first section comprising hydrocodone 30 mg and acetaminophen 3,000 mg;
a second section comprising hydrocodone 25 mg and acetaminophen 2,500 mg;
a third section comprising hydrocodone 20 mg and acetaminophen 2,000 mg;
a fourth section comprising hydrocodone 20 mg and acetaminophen 2,000 mg;
a fifth section comprising hydrocodone 15 mg and acetaminophen 1,500 mg;
a sixth section comprising hydrocodone 15 mg and acetaminophen 1,500 mg;
a seventh section comprising hydrocodone 15 mg and acetaminophen 1,500 mg; and
an eighth section comprising hydrocodone 15 mg and acetaminophen 1,500 mg.

28. The package of claim 27, wherein the package further comprises a rescue portion comprising unit dosage forms comprising hydrocodone 35 mg and acetaminophen 3,500 mg.
